(19) United States
(12) Patent Application Publication
(10) Pub. No.: US 2005/0208109 A1
(43) Pub. Date: Sep. 22, 2005

(54) Non-glue based moisturizing device

(76) Inventor: Mina Houtan, Egg Harbor Township, NJ (US)

Correspondence Address: LAW OFFICES OF KHALILIAN SIRA, LLC, 9100 PERSIMMON TREE ROAD, POTOMAC, MD, 20854, US

(21) Appl. No.: 11/043,100

(22) Filed: Jan. 27, 2005

Related U.S. Application Data
(60) Provisional application No. 60/539,568, filed on Jan. 29, 2004.

(51) Int. Cl. A61K 7/06; A61K 7/11; A61K 9/70

(52) U.S. Cl. 424/443; 424/70.14

(57) ABSTRACT

A non-glue-based moisturizing device for moistening surfaces that are pre-coated with a dry adhesive. The moisturizing device of the invention includes a body that stores an aqueous solution, a tip capable of absorbing, holding and applying the aqueous solution to the pre-coated adhesive surface of the object, and a cap that covers and protects the tip when not in use. The aqueous solution contains water as a major constituent, does not contain colorants, and has a viscosity greater than water.

### I. FIELD OF THE INVENTION

The invention relates to the field of stationeries. Specifically, the invention relates to non-glue based moisturizing devices for moistening objects that are pre-treated with adhesive compounds.

### II BACKGROUND OF THE INVENTION

Numerous devices are described in the prior art that purport to apply adhesive or non-adhesive compounds to a variety of objects. Generally these devices include a housing to store the compound and a tip properly designed to apply the compounds to different surfaces.

For example, U.S. Pat. No. 5,622,560 to Haroutel et al. describes a device for moistening labels that have one face coated with adhesive. The moistening element of the device is a rigid bar whose top portion is split up by a plurality of capillarity slits. Water is used as the moisturizing agent.

EP0-293316 describes a device for moisturizing pre-glued paper through the action of two motorized drive rollers. The lower glued face is in contact with a layer of wires, partially submerged in the water.

U.S. Pat. No. 4,913,946 to Sala et al. describes a fluorescent adhesive tape for use as a highlighter to apply to a support member consisting of a transparent or semi-transparent film containing a layer of adhesive having low adhesive power.

The prior art devices, however, use complex mechanical devices or motors to apply the adhesive substances or water to different surfaces. Additionally, a vast majority of the prior art devices are too rigid and cumbersome to use and do not allow dexterity. The present invention as described herein provides an inexpensive moisturizing device that does not have the aforementioned drawbacks, is efficient, simple, easy to use and inexpensive.

### III. SUMMARY OF THE INVENTION

The invention, as described herein, provides a moisturizing device for moistening an object that is coated with a dry adhesive on one or more surfaces. The moisturizing device of the invention comprises a body that stores an aqueous solution, a tip capable of absorbing, holding and applying the aqueous solution to the surface of the object, and a cap that covers and protects the tip when not in use. The aqueous solution contains water as a major constituent, does not contain colorants, and has a viscosity greater than water.

The viscosity of the aqueous solution is adjusted by addition of organic and/or inorganic compounds. Any compound that is soluble in water and increases the viscosity of water is intended to be encompassed within the scope of the aqueous solution of the invention.

In one embodiment, the aqueous solution contains water along with electrolytes, ions, inorganic materials, or a combination thereof.

In another embodiment, the aqueous solution comprises about 97-99% water, about 0.15-0.64% total protein (e.g., albumin, and/or mucin, among others), about 8-40 mMol/l potassium, about 5-100 mMol/l sodium; about 1.5-2 mMol/l calcium, about 5.5-14 mMol/l phosphate, about 5-70 mMol/l chloride, and about 3-15 mg/100 ml lipids.

In yet another embodiment, the concentration of the dry substance in the aqueous solution is about 1 to about 10 μl, about 2 to about 5 g/l or about 3 to about 4 g/l w/v.

In another embodiment, the aqueous solution additionally contains an antibacterial and/or an antifungal agent.

In yet another embodiment, the moisturizing device is used to moisten dry coated adhesive paper object such as, for example, envelopes, stamps, labels, or a combination thereof.

In another embodiment, the body of the moisturizing device of the invention is made of a clear or transparent plastic to enable a consumer to clearly visualize the level of the aqueous solution within the device.

In a further embodiment of the invention the tip of the moisturizing device is trapezoidal, or round and is made from natural or synthetic fibers including sponge, cellulose; paper, plastic, or rubber, among others.

### IV. BRIEF DESCRIPTION OF THE FIGURES

FIG. 1 is a perspective view of an exemplary embodiment of the moisturizing device of the invention.

FIG. 2 is perspective view of another exemplary embodiment of the moisturizing device of the invention.

### V. DETAILED DESCRIPTION OF THE INVENTION

The invention provides non-glue based moisturizing devices that are uniquely designed and formulated to moisten the adhesive-coated surfaces in a uniform manner, with utmost efficiency, precision and without mess. In particular, the invention provides moisturizing devices for uniformly moistening a paper object that has a light coating of adhesive thereon. The paper objects of the invention include stationery objects such as, for example, pre-coated envelopes, stamps, and labels, among others.

There is a great need in the stationery market for a simple and efficient moisturizing device to moisten dry coated adhesive objects such as envelopes. Envelopes are either coated with a dry adhesive or are self sealing. Self-sealing envelopes are coated with a wet adhesive and would seal without the need for moisturizing. There is, however, a shortage of self-sealing envelopes, and especially self-sealing colored envelopes, in the market. Additionally, the price of the self-sealing envelopes is twice as much as the dry coated adhesive envelopes. Accordingly, the novel moisturizing device of the invention is economically and practically attractive to the majority of businesses and private activities that send out vast amounts of mail daily.

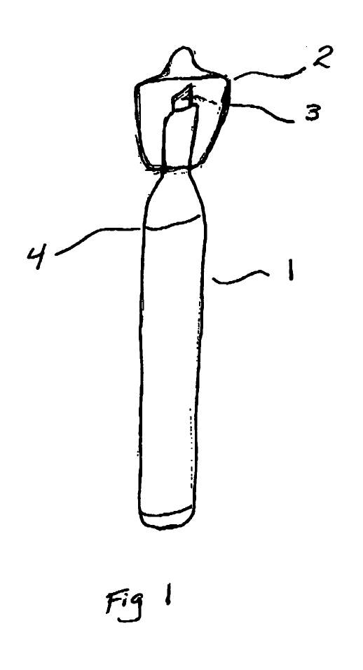

Referring to FIG. 1, the moisturizing device of the invention comprises three compartments, a body portion 1, a cap portion 2, and a tip portion 3. The body portion 1 is filled with an aqueous solution to moisten a pre-coated adhesive on an object. The cap portion 2 covers and protects the tip. Accordingly, the shape of the cap is controlled by the shape of the tip.

The body, cap and the tip of the device can be made in a variety of shapes, including cylindrical, oblong, elliptical, oval, round, square, and other shapes, so long as the device can be comfortably held in the hand and used for its intended purpose. A preferred body and cap shape for the moisturizing device of the invention is a cylindrical shape.

The body and cap are made of, for example, plastic, wood, metal, and other natural or synthetic materials. A preferred material to use is plastic (e.g., thermoplastic). In one embodiment, the body is made of a transparent plastic that shows the level of aqueous solution therein.

The tip has the capacity of absorbing, holding and releasing liquids. The tip is intended not only to apply the aqueous solution but also to accommodate itself to the varying configurations and sizes of the pre-treated adhesive objects that are subjected to the moisturizing device of the invention. Accordingly, the tip is formed in a variety of shapes according to its intended use.

In an exemplary embodiment, shown in FIG. 1, the tip has a trapezoidal shape similar to the shape of the tip of a regular highlighter. The trapezoidal tip has, for example, a high point and a low point. The high point of the tip is, for example, from about 0.25 cm, to about 1 cm. The low point of the tip is, for example, from about 0.12 cm to about 0.50 cm. The thickness of the tip is for example, from about 0.1 cm to about 1 cm.

It is intended herein that specific values recited also include the range of values between the two values recited. For example, it is intended herein that values of about 0.25 cm and about 0.50 cm, also encompass 0.30, 0.35, 0.40, 0.45, cm, etc., without actually reciting each specific value therein.

In one embodiment, the high point of the tip is about twice as high as the low point of the tip.

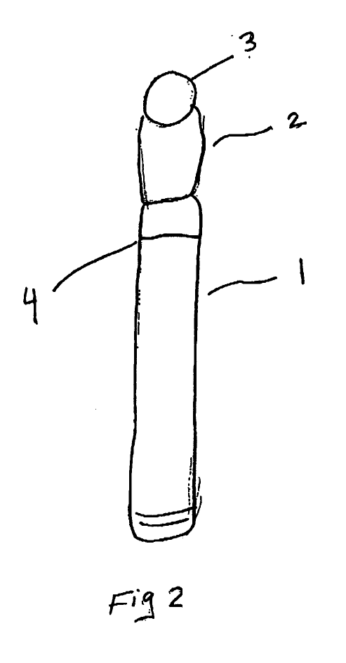

Referring to FIG. 2, another exemplary embodiment of the moisturizing device of the invention is shown wherein the tip is in a round shape and can rotate in a circular fashion within the body of the device.

The tip is made from, for example, sponge-like pliable materials that can absorb and release liquids with minimum pressure. The sponge-like pliable materials of the tip are made of, for example, natural or synthetic fiber, such as for example, cellulose material (e.g., paper), plastic, rubber, sponge, brush, or a combination thereof, among others. In one embodiment, the tip is made of a thermoplastic material.

In an exemplary embodiment, the moisturizing device of the invention resembles an ordinary highlighter. The shape and structure of the tip of the moisturizing device is the same as the tip of an ordinary highlighter, which can easily absorb and release liquid in a precise and clean format.

In one embodiment, the moisturizing device is accompanied with one or more refills to replenish the moisturizing device when it is dried out. The refills are either placed as a whole inside the body of the moisturizing device, or their contents are transferred to the body of the moisturizing device.

The body of the device is filled with an aqueous solution. The aqueous solution contains water as a major constituent, and does not contain colorants. Colorants, as used herein, refer to any compound or composition that adds color to the solution. Colorants, within the scope of the invention, encompass dyes, including fluorescent and non-fluorescent dyes, and various stains, among others. The viscosity of the aqueous solution is preferably greater than water.

The water content of the aqueous solution can range, for example, from about 90% to about 99%. In one exemplary embodiment, the water content of the aqueous solution is from about 97% to about 99.9%. In another exemplary embodiment, the aqueous solution comprises tab water or distilled water.

The minor constituents of the aqueous solution of the moisturizing device comprise, for example, one or more of the following: electrolytes, ions (e.g., sodium, potassium, chloride, and calcium, among others), inorganic materials (e.g., proteins, lipids, carbohydrates), bactericidal, or bacteriastatic agents, among others.

In one embodiment, the aqueous solution of the invention is sterilized (e.g., by autoclave or filter sterilization). The sterilized aqueous solution is transferred under sterile conditions to the moisturizing device. The moisturizing device may additionally contain a tube that can be pre-sterilized to accept the aqueous solution. In those embodiments where the moisturizing device can not be sterilized by autoclave, the moisturizing device is treated with anti-bacterial and/or anti-fungal agents prior to the transfer of the aqueous solution therein.

In another embodiment, the aqueous solution additionally contains an antibacterial/and or an antifungal agent to reduce the chance of contamination.

Numerous variations in the construction of the moisturizing device of this invention, within the scope of the appended claims, will occur to those skilled in the art, in the light of the foregoing disclosure.

1. A moisturizing device for moistening an object having a surface that is coated with a dry adhesive, the moisturizing device comprising, a body that stores an aqueous solution, a tip capable of absorbing, holding and applying the aqueous solution to the surface of the object, and a cap that covers and protects the tip when not in use, wherein the aqueous solution contains water as a major constituent, does not contain colorants, and has a viscosity greater than water.

2. The moisturizing device of claim 1, wherein the aqueous solution additionally contains electrolytes, ions, inorganic materials, or a combination thereof.

3. The moisturizing device of claim 2, wherein the aqueous solution comprises about 97-99% water, about 0.15-0.64% total protein, about 8-40 mMol/l potassium, about 5-100 mMol/l sodium; about 1.5-2 mMol/l calcium, about 5.5-14 mMol/l phosphate, about 5-70 mMol/l chloride, and about 3-15 mg/100 ml lipids.

4. The moisturizing device of claim 1, wherein the aqueous solution contains about 1 to about 10 g/l w/v of a dry substance.

5. The moisturizing device of claim 1, wherein the aqueous solution contains about 2 to about 5 g/l w/v of a dry substance.

6. The moisturizing device of claim 1, wherein the aqueous solution contains about 3 to about 4 g/l w/v of a dry substance.

7. The moisturizing device of claim 1, wherein the object is made of paper.

8. The moisturizing device of claim 7, wherein the object comprises, envelopes, stamps, labels, or a combination thereof.

9. The moisturizing device of claim 1, wherein the aqueous solution additionally contains an antibacterial and/or an antifungal agent.

10. The moisturizing device of claim 1, wherein the body is made of a transparent plastic.

11. The moisturizing device of claim 1, wherein the tip is trapezoidal.

12. The moisturizing device of claim 1, wherein the tip is round.

13. The moisturizing device of claim 1, wherein the tip is made from a natural or synthetic brush.

14. The moisturizing device of claim 11, wherein the tip has a high point from about 0.25 cm, to about 1 cm long and a low point from about 0.12 cm to about 0.50 cm long, and a thickness of from about 0.1 cm to about 1 cm.

15. The moisturizing device of claim 12, wherein the high point of the tip is about twice as high as the low point of the tip.

17. The moisturizing device of claim 1, wherein the tip is made from sponge, natural or synthetic fiber, cellulose; paper, plastic, or rubber.
